(19) United States
(12) Patent Application Publication
(10) Pub. No.: US 2006/0205632 A1
(43) Pub. Date: Sep. 14, 2006

(54) Pro-fragrance and pro-flavorant compositions

(76) Inventor: Luca Turin, London (GB)

Correspondence Address: MILES & STOCKBRIDGE PC, 1751 PINNACLE DRIVE, SUITE 500, MCLEAN, VA, 22102-3833, US

(21) Appl. No.: 11/335,827

(22) Filed: Jan. 20, 2006

Related U.S. Application Data
(60) Provisional application No. 60/651,078, filed on Feb. 9, 2005.
(60) Provisional application No. 60/661,505, filed on Mar. 15, 2005.
(60) Provisional application No. 60/668,107, filed on Apr. 5, 2005.
(60) Provisional application No. 60/724,778, filed on Oct. 11, 2005.

(51) Int. Cl. A61K 8/49; C11D 3/50; C07C 249/02; C07D 309/30; C07D 307/34

(52) U.S. Cl. 512/11; 424/65; 510/101; 549/293; 549/321; 564/277; 564/278; 512/20

(57) ABSTRACT

A pro-odorant or pro-flavorant having the formula: 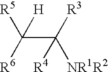 the substituents being such that the compound is an odorant or flavorant; or the formula:
R⁹R¹⁰C═NR¹¹
the substituents being such that a compound of formula R⁹R¹⁰C═O is an odorant or flavorant; or the formula: 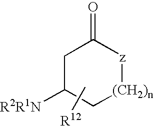
the substituents being such that a compound of formula: 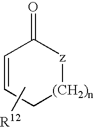
an odorant or flavorant; or formula (IV):

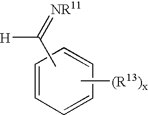

The present invention relates generally to the field of fragrances and flavorants. More particularly, the present invention relates to novel pro-fragrances and pro-flavorants that release their fragrance and flavor characteristics over extended times and their use as fragrance and flavor compositions for application to a variety of substrates.

Perfumed and flavored products, compositions and articles are well known in the art and widely used. The acceptance of these products by consumers depends to a large extent, however, on the ability thereof to retain and release their fragrance and/or flavor over time. The inability of certain perfume components to maintain their fragrance characteristics for acceptable lengths of time has been an ongoing problem in the area of fabric care. Perfume additives have traditionally been included in fabric detergents, bleaching compositions and fabric softeners. Typically, however, such perfumes are quickly lost to the fabric treatment environment, e.g. the wash and rinse water cycles in washing machines and the drying cycles in clothes dryers and little if any is imparted to or remains long on the treated fabric. Such inefficient delivery of expensive perfume components in fabric treatments results in high costs to manufacturers and consumers alike and has an adverse effect on consumer acceptance of such products.

The desire to extend the fragrance- and flavor-releasing properties over time of fragrance and flavor compositions exists in other industries and applications as well. For example, the consumer acceptance of numerous products would be greatly increased and their costs greatly reduced if the fragrance and flavor components incorporated therein could be made to last longer. Such products include foods, personal care products, household and industrial cleaning compositions, disinfectants, beverages, chewing gums, pharmaceutical and medicinal compositions and orally-deliverable matrices.

Numerous efforts have been made to improve the “lasting” properties of fragrances, particularly in the fabric care industry

U.S. Pat. No. 5,188,753 describes carrier mechanisms, such as encapsulation, which has not proven to be successful.

Other solutions involve the preparation of reaction products of the perfume with reactants to form products (pro-fragrances) that provide a delayed release of the fragrance over a longer period of time than by the use of the fragrance itself. See U.S. Patent Application Publication Nos. 20030211963, 20030211960, 20030134772, 20030073607, 20050043205, 20050009727, 20040147426, 20040116320, 20040097397, 20040018955 and U.S. Pat. Nos. 6,858,575; 6,790,815; 6,764,986; 6,740,713; 6,699,823; 6,566,312; 6,511,948; 6,451,751 and 6,413,920. Although providing compositions with pro-fragrance characteristics, the reaction of the fragrance compound with the reagents identified in the above-mentioned patents and publications provide reaction products that are either solids or comprise fluids with high viscosities.

It has also been suggested in the prior art to form Schiff bases of aldehydic fragrance compounds or aromachemicals. However, in such cases volatile amines such as alkyl anthranilates have been utilized that impart their own, sometimes objectionable, odor to the environment when the Schiff base degrades, thereby adversely impacting on the fragrance characteristics of the aromachemical.

The listing or discussion of a prior-published document in this specification should not necessarily be taken as an acknowledgement that the document is part of the state of the art or is common general knowledge.

It is an object of the invention to provide novel pro-fragrance and pro-flavorant compositions having significantly lower viscosities than those of the prior art.

The present invention provides a pro-odorant or pro-flavorant that is liquid at room temperature and has a relatively low viscosity and has the formula (I): 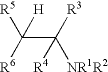
wherein R¹ is H, an aliphatic group or an aromatic group, R² is an aliphatic group or an aromatic group, provided that the total number of carbon atoms in the groups R¹ and R² is 10 or more; R⁵ is CN, COOH, COOR⁷, CHO or C(O)R⁸; R³, R⁴ and R⁶ are each independently hydrogen or organic moieties which together with R⁵ render a compound of formula R⁵R⁶C═CR³R⁴ a material having odorant or flavourant characteristics and R⁷ and R⁸ are each independently an organic moiety.

The groups R³, R⁴ and R⁶ may for example be H, linear or branched or cyclic C₁₋₁₂ alkyl, alkenyl or alkoxy, substituted linear or branched or cyclic C₁₋₁₂ alkyl, alkenyl or alkoxy, substituted or unsubstituted aromatic, halo, hydroxyl, thiol, thioether, amine, carboxylic acid, ester, nitro, cyano, isocyano, sulfonic acid, urea and thiourea. Suitable substituents on the substituted alkyl, alkenyl, alkoxy or aromatic groups include halo, hydroxyl, thiol, thioether, amine, carboxylic acid, ester, nitro, cyano, isocyano, sulfonic acid, urea and thiourea.

The groups R⁷ and R⁸ may for example be linear or branched or cyclic C₁₋₁₂ alkyl, alkenyl or alkoxy, substituted linear or branched or cyclic C₁₋₁₂ alkyl, alkenyl or alkoxy, substituted or unsubstituted aromatic, halo, hydroxyl, thiol, thioether, amine, carboxylic acid, ester, nitro, cyano, isocyano, sulfonic acid, urea and thiourea. Suitable substituents on the substituted alkyl, alkenyl, alkoxy or aromatic groups include halo, hydroxyl, thiol, thioether, amine, carboxylic acid, ester, nitro, cyano, isocyano, sulfonic acid, urea and thiourea.

Preferably at least one of R⁴ and R⁶ is H, for example both of R⁴ and R⁶ may be H. Another preferred group for R⁶ is cyclohexyl.

In one aspect of the invention R¹ is H, alkyl, alkenyl or alkoxy, R² is alkyl, alkenyl or alkoxy, provided that the total number of carbon atoms in the groups R¹ and R² is 10 or more, preferably 12 to 25, for example 15 to 20 carbon atoms, eg 18 carbon atoms. The alkyl, alkenyl or alkoxy group may be straight chained or branched or cyclic. Preferably R¹ is H. Alternatively, at least one of R¹ and R² may be an aromatic group.

Particularly preferred compounds of formula (I) include:

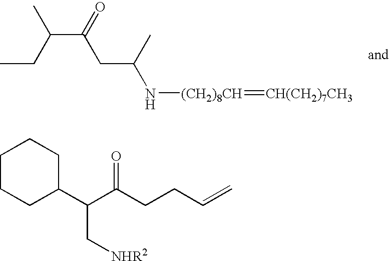

The present invention also provides a pro-odorant or pro-flavorant that is liquid at room temperature and has a relatively low viscosity and has the formula (II):
R⁹R¹⁰C═NR¹¹
wherein R¹¹ has at least 10 carbon atoms and is an aliphatic group or an aromatic group; R⁹ and R¹⁰ are each independently H or organic moieties which together with C═O render a compound of formula R⁹R¹⁰C═O a material having odorant or flavourant characteristics, provided that only one of R⁹ and R¹⁰ is hydrogen.

The groups R⁹ and R¹⁰ may for example be H, linear or branched or cyclic C₁₋₁₂ alkyl, alkenyl or alkoxy, substituted linear or branched or cyclic C₁₋₁₂ alkyl, alkenyl or alkoxy, substituted or unsubstituted aromatic, halo, hydroxyl, thiol, thioether, amine, carboxylic acid, ester, nitro, cyano, isocyano, sulfonic acid, urea and thiourea. Suitable substituents on the substituted alkyl, alkenyl or alkoxy groups or aromatic include aromatic groups, halo, hydroxyl, thiol, thioether, amine, carboxylic acid, ester, nitro, cyano, isocyano, sulfonic acid, urea and thiourea.

Preferably R⁹ and R¹⁰ are each independently H, linear or branched or cyclic C₁₋₁₂ alkyl, alkenyl or alkoxy, substituted linear or branched C₁₋₁₂ alkyl, alkenyl or alkoxy substituted or unsubstituted aromatic, more preferably linear or branched C₁₋₆ alkyl or alkenyl. For example R⁹ may be an alkyl group containing four carbon atoms such as —CH(CH₃)CH₂CH₃ or may contain an aromatic group, for example CH₂CH(CH₃)(C₆H₅). For example R¹⁰ may be hydrogen or a methyl group.

In one aspect of the invention R¹¹ has at least 10 carbon atoms and is alkyl, alkenyl or alkoxy, preferably 12 to 25, for example 15 to 20 carbon atoms, eg 18 carbon atoms. The alkyl, alkenyl or alkoxy group may be straight chained or branched.

A preferred compound of formula (II) is:

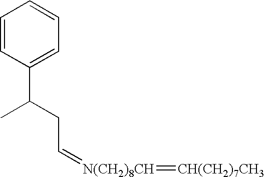

The present invention also provides a pro-odorant or pro-flavorant that is liquid at room temperature and has a relatively low viscosity and has the formula (III): 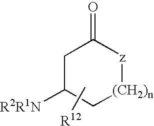
wherein R¹ is H, an aliphatic group or an aromatic group, R² is an aliphatic group or an aromatic group, provided that the total number of carbon atoms in the groups R¹ and R² is 10 or more; Z is CH₂ or 0, n is 0 or 1, such that the ring is a 5 or 6 membered ring, R¹² is alkyl, alkenyl or alkoxy having up to 10 carbon atoms so as to render a compound of formula 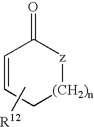
   a material having odorant or flavorant characteristics.

In one aspect of the invention R¹ is H, alkyl, alkenyl or alkoxy, R² is alkyl, alkenyl or alkoxy, provide that the total number of carbon atoms in the groups R¹ and R² is 10 or more, preferably 12 to 25, for example 15 to 2, eg 18. The alkyl, alkenyl or alkoxy group may be straight chained or branched. More preferably R¹ is H. Alternatively, at least one of R¹ and R² may be an aromatic group.

R¹² may be a straight chain or branched alkyl or alkenyl or alkoxy. Optionally, R¹² may be substituted. Suitable substituents include halo, hydroxyl, thiol, thioether, amine, carboxylic acid, ester, nitro, cyano, isocyano, sulfonic acid, urea and thiourea.

Compounds of formula (III) in which Z is O and/or n is 0 are preferred.

Particularly preferred compounds of formula (III) include:

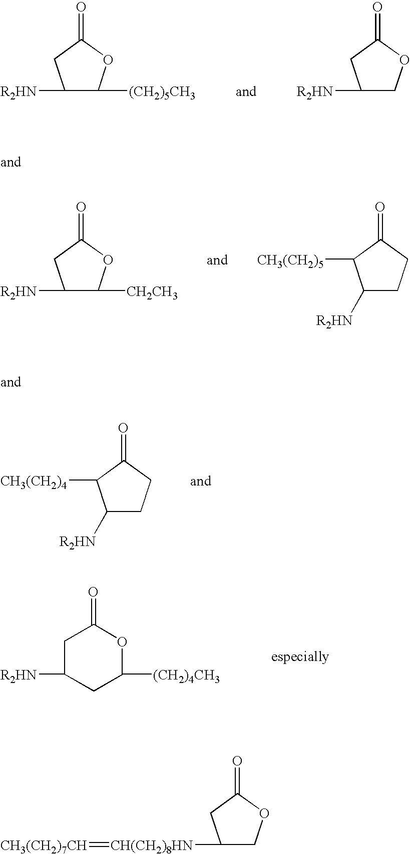

The present invention also provides a pro-odorant or pro-flavorant that is liquid at room temperature and has a relatively low viscosity and has the formula (IV): 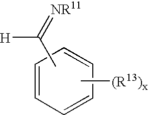
wherein R¹¹ has at least 10 carbon atoms and is an aliphatic group or an aromatic group; the or each R¹³ is independently a straight or branched chain, saturated or unsaturated hydrocarbyl group or alkoxy group having from 1 to 8 carbon atoms or two groups R¹³ together with the carbon atoms to which they are attached form a five or six membered ring which may be saturated or unsaturated (including aromatic) and which may be optionally substituted with from 1 to 3 alkyl groups having from 1 to 6 carbon atoms; and x is from 1 to 5 so as to render a compound of formula 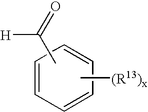
a material having odorant or flavorant characteristics.

Preferably at least one R¹³ is an alkyl, alkenyl or alkoxy group or two groups R¹³ together with the carbon atoms to which they are attached form a five or six membered ring. Preferred substitutents on a ring formed by two groups R¹³ and the carbon atoms to which they are attached have from 1 to 4 carbon atoms. For example, at least one R¹³ may be an alkyl, alkenyl or alkoxy group, which is straight chained or branched and has from 1 to 4 carbon atoms. In particular, at least one R¹³ may be t-butyl and/or methoxy. In a preferred aspect, up to three groups R¹³ can be t-butyl and optionally one or more groups R¹³ can be methoxy.

A preferred compound of formula (IV) is:

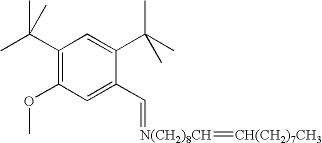

As used herein, by the terms “pro-odorant” and “pro-flavorant” we mean compounds which themselves have substantially no odor or flavor but degrade over time and/or under certain conditions to provide the odorant or flavorant molecule on which they are based and thus provide the odor or flavor of that base molecule.

The compounds of the invention of formula (I) may be produced by a Michael addition reaction of an aromachemcial comprising a carbon-carbon double bond in 1,4 conjugation with an aldehyde or ketone group such as an α,β-unsaturated ketone with a primary or secondary amine. The aromachemcials suitable for use in this reaction have the formula (V): 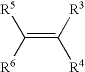
wherein R³, R⁴, R⁵ and R⁶ are each as defined above.

The amines used in this reaction have the formula NHR¹R² wherein R¹ and R² are as defined above.

A typical reaction profile is as follows:

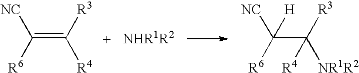

The compounds of the invention of formula (II) may be produced by the reaction of an aromachemical having at least one aldehyde or ketone group with a primary amine to form an imine of formula (II).

The aldehydes and ketones that may be used in this reaction have the formula (VI):
R⁹R¹⁰C═O
wherein R⁹ and R¹⁰ are as defined above.

The amines that may be used in this reaction have the formula NH₂R¹¹, wherein R¹¹ is a defined above.

A typical reaction profile, employing primary amines (the products being called azomethines, Schiff bases or the more preferred name imines), is as follows:

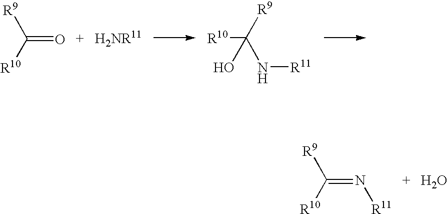

The compounds of the invention of formula (III) may be produced by a Michael addition reaction of a primary or secondary amine with an α,β-unsaturated ketone having aromachemical/flavorant characteristics of the formula (VII): 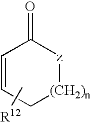
wherein Z is CH₂ or O, n is 0 or 1, such that the ring is 5- or 6-membered, respectively, and R¹² is as defined above.

The amines used in this reaction have the formula NHR¹R², wherein R¹ and R² are as defined above.

A typical reaction profile is as follows:

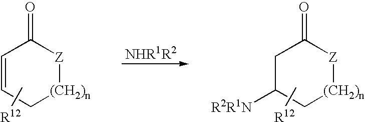

The compounds of the invention of formula (IV) may be produced by the reaction of an aromachemical having at least one aldehyde group with a primary amine to form an imine of formula (IV).

The aldehydes that may be used in this reaction have the formula (VIII): 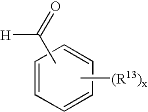
wherein R¹³ and x are as defined above.

A typical reaction profile, employing primary amines (the products being called azomethines, Schiff bases or the more preferred name imines), is as follows:

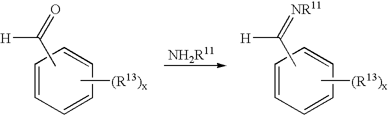

For example, a compound of formula: 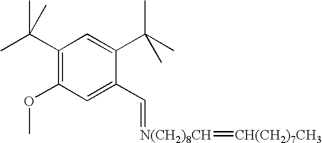
may be prepared by reacting a compound of formula 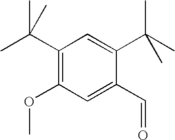
(2,4-di-t-butyl,5-methoxybenzaldehyde) with H₂N(CH₂)₈CH═CH(CH₂)₇CH₃.

The amines that may be used in this reaction have the formula NH₂R¹¹, wherein R¹¹ is as defined above.

By means of these simple, well known and conventional methods, compounds and compositions containing the compounds of the invention can be made. The reaction conditions and parameters for carrying out these procedures are well known in the prior art, although the requirements in a particular reaction for the production of optimum results may depend in each case on the reactants employed.

The perfume ingredient (ie the compound of formula (V), (VI), (VII) or (VIII)) is typically used in equimolar amount to the amine compound so as to enable the reaction to take place and provide the resulting amine reaction product. Of course, higher amounts are not excluded and may even be preferred when the amine compound comprises more than one amine function. When the amine compound has more than one free primary and/or secondary amine function, several different perfume raw materials can be linked to the amine compound.

The amine compounds used in the present invention typically have an Odor Intensity Index of less than that of a 1% solution of methylanthranilate in dipropylene glycol, and a Dry Surface Odor Index of more than 5.

To measure the Odor Intensity Index, it is meant that the pure chemical is diluted at 1% in dipropylene glycol, an odor-free solvent used in perfumery. This percentage is more representative of usage levels. Smelling strips or so called “blotters”, are dipped and presented to the expert panelist for evaluation. Expert panelists are assessors trained for at least six months in odor grading and whose gradings are checked for accuracy and reproducibility versus a reference on an on-going basis. For each amine compound, the panelist is presented with two blotters: one reference methylanthranilate) and the sample. The panelist is asked to rank both smelling strips on the 0 to 5 odor intensity scale, 0 being no odor detected and 5 being very strong odor present.

Suitable amines for use in the reactions to produce the compounds of the invention are preferably non-fragrant, odorless, non-volatile amines having a relatively low vapor pressure and high molecular weight, i.e. aromatic or aliphatic amines containing more than about 10 carbon atoms. Preferably the amines have a molecular weight of at least 150 daltons.

Suitable amines for use in the present invention include odourless, low vapour pressure aliphatic or aromatic amines containing at least one free, unmodified primary and/or secondary amino group. Any suitable alkyl, alkenyl or alkoxy, branched or straight chain amine having a total of at least 10 carbon atoms that is relatively odourless and forms a relatively insoluble derivative with the aromachemical that has a relatively low viscosity may be employed in the practice of the invention. Suitable amines include but are not limited to n-dodecylamine, n-tetradecylamine, n-hexadecylamine, n-octadecylamine, oleylamine, cocoalkylamines, soyaalkylamines, tallowalkylamines, hydrogenated tallowalkylamines, di-n-hexylamine, di-n-octylamine, di-n-decylamine, di-n-docecylamine, di-n-tetradecylamine, di-n-hexadecylamine, di-n-octadecylamine, dioleylamine, dicocoalkylamines, disoyaalkylamines, ditallowalkylamines, di-(hydrogenated tallowalkyl)amines, mixed secondary amines such as n-dodecylmethylamine, n-tetradecylmethylamine, n-hexadecylmethylamine, n-octadecylmethylamine, oleylmethylamine, cocoalkylmethylamines, soyaalkylmethylamines, tallowalkylmethylamines, hydrogenated tallowalkylmethylamines, n-decylethylamine, n-dodecylethylamine, n-tetradecylethylamine, n-hexadecylethylamine, n-octadecylethylamine, oleylethylamine, cocoalkylethylamines, soyaalkylethylamines, tallowalkylethylamines, hydrogenated tallowalkylethylamines, branched isomers and/or derivatives thereof and mixtures thereof.

By “primary and/or secondary amine”, it is meant a component that carries at least one primary and/or secondary amine and/or amide function.

A typical disclosure of ketones and aldehydes that are traditionally used in perfumery and as flavourings and that would be suitable for use in the preparation of the compounds of the present invention can be found in “Perfume and Flavour Chemicals”, Vol. I and II, S. Arctander, Allured Publishing, 1994, ISBN 0-931710-35-5.

Suitable ketone and aldehyde aromachemicals, fragrances or flavorants that may be used to prepare the compositions of the invention include most if not all ketone and aldehyde aromachemicals, fragrances or flavorants known in the art. Examples of the ketone and aldehyde aromachemicals, fragrances or flavorants that may be used to prepare the compounds of the invention include but are not limited to buccoxime; iso jasmone; methyl beta naphthyl ketone; musk indanone; tonalid/musk plus; alpha-damascone, beta-damascone, delta-damascone, iso-damascone, damascenone, damarose, methyl-dihydrojasmonate, menthone, carvone, camphor, fenchone, alpha-ionone, beta-ionone, gamma-methyl so-called Ionone, fleuramone, dihydrojasmone, cis-jasmone, iso-E-Super, methyl-cedrenyl-ketone or methyl-cedrylone, acetophenone, methyl-acetophenone, para-methoxy-acetophenone, methyl-beta-naphtyl-ketone, benzyl-acetone, benzophenone, para-hydroxy-phenyl-butanone, celery ketone or livescone, 6-isopropyldecahydro-2-naphtone, dimethyl-octenone, freskomenthe, 4-(1-ethoxyvinyl)-3,3,5,5,-tetramethyl-cyclohexanone, methyl-heptenone, 2-(2-(4-methyl-3-cyclohexen-1-yl)propyl)-cyclopentanone, 1-(p-Menthen-6(2)-yl)-1-propanone, 4-(4-hydroxy-3-methoxyphenyl)-2-butanone, 2-acetyl-3,3-dimethyl-norbornane, 6,7-dihydro-1,1,2,3,3-pentamethyl-4(5H)-indanone, 4-damascol, dulcinyl or cassione, gelsone, hexalon, isocyclemone E, methyl cyclocitrone, methyl-lavender-ketone, orivon, para-tertiary-butyl-cyclohexanone, verdone, delphone, muscone, neobutenone, plicatone, veloutone, 2,4,4,7-tetramethyl-oct-6-en-3-one, tetrameran, adoxal, anisic aldehyde, cymal, ethyl vanillin, florhydral, helional, heliotropin, hydroxycitronellal, koavone, lauric aldehyde, lyral, methyl nonyl acetaldehyde, P. T. bucinal, phenyl acetaldehyde, undecylenic aldehyde, vanillin, 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)propanal, 2-methyl-3-(para-methoxyphenyl)propanal, 2-methyl-4-(2,6,6-trimethyl-2(1)-cyclohexen-1-yl)butanal, 3-phenyl-2-propenal, cis/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclo-hexene-1-carboxaldehyde, 5 or 6 methoxyohexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclohexene-carboxaldehyde, 7-hydroxy-3,7-dimethyl-octanal, trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl)-benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, hexahydro-8,8-dimethyl-2-naphthaldehyde, 3-propyl-bicyclo[2.2.1]hept-5-ene-2-carbaldehyde, 9-decenal, 3-methyl-5-phenyl-1-pentanal, methylnonyl acetaldehyde, hexanal, trans-2-hexenal, 1-para-menthene-q-carboxaldehyde, 2-methyl-2-(para-isopropylphenyl)-propionaldehyde, 1-(2,6,6-trimethyl-2-cyclohexan-1-yl)-2-buten-1-one and/or para-methoxy-acetophenone, undecylenic aldehyde, undecalactone gamma, heliotropin, dodecalactone gamma, para-anisic aldehyde, para-hydroxy- to phenyl-butanone, cymal, ionone alpha, damascenone, ionone beta, methyl-nonyl ketone, compounds having the formula: 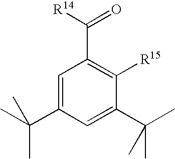
wherein R¹⁴ and R¹⁵ are, independently, H or straight or branched chain alkyl or alkenyl or alkoxy having 1 to 6 carbon atoms, or mixtures thereof.

Suitable aldehydic musks for derivitization according to the present invention include the aromatic (benzenic), and polycyclic musks. The benzenic musks suitable for the present invention include 2,4-di-tertiarybutyl-5-methoxybenzaldehyde. An exemplary benzenic aldehydic musk is 2,4-ditertiarybutyl-5-methoxybenzaldehyde, having the structure:

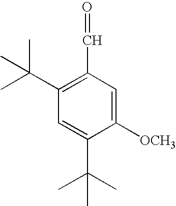

Suitable polycyclic musks are those having the structures: 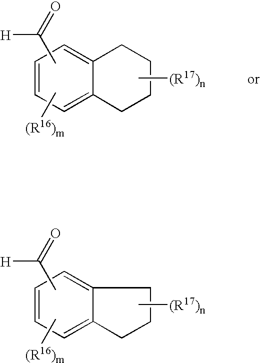
wherein R¹⁶ and R¹⁷ may be the same or different and are a straight or branched chain, saturated or unsaturated hydrocarbyl group; preferably, alkyl or alkenyl, having 1 to 8 carbon atoms, and m is 1 to 4 and n is 1 to 6.

Typical such polycyclic musks are those having the structures:

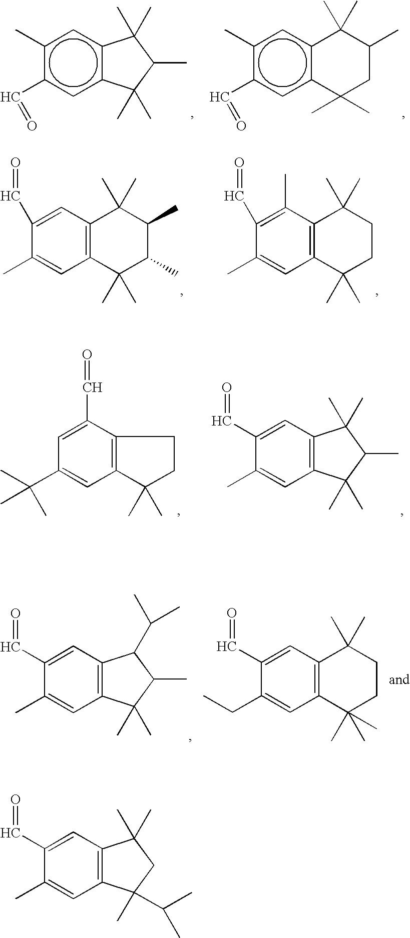

Examples of unsaturated aromachemicals, fragrances and flavorants that may be employed to prepare the compounds of the invention include 4-methyl-pentan-2-ol-crotonate, 1-cyclohexyl-ethyl-crotonate (Datilat) and hexylcrotonate; butyl pentenoate; ethyl pentenoate; hexyl angelate; hexyl pentenoate; iso-amyl angelate; iso-butyl angelate; iso-amyl pentenoate; iso-byutul pentenoate; methyl allyl pentenoate; methylgeranate; cis-3-hexenylsalicylate; methyl-2-nonenoate; 3,7-dimethyl-6-octenyl-2-methylcrotonate; phenylethyl cinnamate; 3,7-dimethyl-2,6-octadienyl-2-methylcrotonate; methyl-2-nonenoate; 4-methyl-pentan-2-ol-crotonate (Frutinat); 2-cyclopentyl-cyclopentylcrotonate (Pyproprunat); 3,7-dimethyl-2(3), 6-nonadienenitrile (lemonile), tridecene-2-nitrile, 3,12-tridecadienenitrile, 3-methyl-5-phenyl-2-pentenenitrile, 3,7-dimethyl-2,6-octadienenitrile and cinnamylnitrile, filbertone, 2-pentyl-2-cyclopenten-1-one, 2-cyclohexyl-1,6-heptadien-3-one, delta-2-decenolactone, 2-pentyl-2-cyclopenten-1-one, 2-hexyl-2-cyclopenten-1-one, 2-hexen-1,4-lactone, but-2-en-1,4-lactone, 2-decen-1,4-lactone, 2-me-2-pentenoic acid or mixtures thereof.

Preferred ketones for use to produce the compounds of the invention include Alpha-Damascone, Delta-Damascone, Iso-Damascone, Carvone, Gamma-Methyl-lonone, Iso-E-Super, 2,4,4,7-Tetramethyl-oct-6-en-3-one, Benzyl Acetone, Beta Damascone, Damascenone, methyl dihydrojasmonate, methyl cedrylone, and mixtures thereof.

Preferred aldehydes for use in the production of the compounds of the invention include 1-decanal, benzaldehyde, florhydral, 2,4-dimethyl-3-cyclohexen-1-carboxaldehyde; cis/trans-3,7-dimethyl-2,6-octadien-1-al; heliotropin; 2,4,6-trimethyl-3-cyclohexene-1-carboxaldehyde; 2,6-nonadienal; alpha-n-amyl cinnamic aldehyde, alpha-n-hexyl cinnamic aldehyde, P. T. Bucinal, lyral, cymal, methyl nonyl acetaldehyde, hexanal, trans-2-hexenal, and mixture thereof.

Generally, the compositions of the present invention are liquid at room temperature (15 to 25° C.) and have a viscosity below about 250 cP, preferably below about 200 cP, when measured at about 20° C. Typically, the preferred compounds of the invention have viscosities below about 100 cP, for example 20 to 100 cP or 50 to 80 cP when measured at about 20° C.

The viscosity can be measured using any suitable method known in the art.

The present invention provides for the use of the compounds of the invention and mixtures thereof as a flavor and/or fragrance.

The present invention also provides compositions, products, preparations or articles having improved aroma, fragrance or odor characteristics containing a compound or mixture of compounds of the invention as described above.

The present invention also provides methods to confer, improve, enhance or modify the taste or flavor property of a composition, product, preparation or article which comprises adding thereto a flavor effective amount of a composition or mixture of compounds of the invention as described above.

A method to confer, improve, enhance or modify the aroma, fragrance or odor characteristics of compositions, products, preparations or articles which comprises adding thereto an aroma, fragrance or odor effective amount of a composition or mixture of compounds of the invention as described above is also provided.

The compounds of the present invention can be used to provide a delayed release of the perfume ingredient. Not to be bound by theory, the release is believed to occur by the following mechanisms: the perfume components are released upon breaking down of the imine or amine bond, leading to the release of the perfume component and of the amine compound. This can be achieved, for example, by hydrolysis, photochemical cleavage, oxidative cleavage, or enzymatic cleavage. Treatment with air moisture and/or water may successfully release the perfume component and the amine compound. However, other means of release are not excluded like hydrolysis, photochemical cleavage, oxidative cleavage, or enzymatic cleavage. Still other means of release for the perfume compounds can be considered such as by the steaming step of ironing the treated fabric, tumble-drying, and/or wearing. The present invention application compositions include compositions where there is a need of a delayed release of an active perfume. This includes compositions for use in the rinse such as softening compositions, personal cleansing such as shower gels, deodorants, bars, shampoos; stand alone compositions such deodorizing compositions, insecticides, etc. Preferred are those compositions, which result in contacting the compound of the invention with fabric. The compositions of the invention are suitable for use in any step of the domestic treatment that is a pre- and/or post-treatment composition, as a wash additive, as a composition suitable for use in the rinse process. Obviously, multiple applications can be made such as treating the fabric with a pre-treatment composition of the invention and thereafter with the composition suitable for use in the rinse process and/or drying process.

By compositions suitable for use in the rinse process, these are to be understood to include compositions such as rinse added fabric softener compositions and dryer added compositions (e.g. sheets) that provide softening and/or antistatic benefits, as well as rinse additives. Preferred are those compositions that result in contacting the compound of the invention with fabric. These are to be understood to include compositions such as rinse added fabric softener compositions and dryer added compositions (e.g. sheets) that provide softening and/or antistatic benefits.

The compounds of the invention can be included in virtually any article of manufacture that can include the non-derivatized fragrance or flavorant compound, or for that matter, other fragrances, whether natural or artificial. Examples include bleach, detergents, flavorings and fragrances, beverages, including alcoholic beverages, and the like. The compounds of the invention can be used in applications like soaps, shampoos, body deodorants and antiperspirants, solid or liquid detergents for treating textiles, fabric softeners, detergent compositions and/or all-purpose cleaners for cleaning dishes or various surfaces, for both household and industrial use. Of course, the use of the compounds is not limited to the above-mentioned products, as they be used in other current uses in perfumery, namely the perfuming of soaps and shower gels, hygiene or hair-care products, as well as of body deodorants, air fresheners and cosmetic preparations, and even in fine perfumery, namely in perfumes and colognes.

The compounds of the invention also find utility in foods, flavorings, beverages such as beer and soda, denture cleansers (tablets), flavored orally-delivered products such as lozenges, candies, chewing gums, matrices, pharmaceuticals and the like. These uses are described in more detail below.

The compounds of the invention can be used as perfuming ingredients, as single compounds or as mixture thereof, preferably in an amount of at least about 30% by weight of the perfume composition, more preferably in an amount of at least about 60% by weight of the composition. The compounds can be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all of the above applications, the compounds of the invention can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through its general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin. A large number of these ingredients described in reference textbooks such as the book of S. Arctander, Perfume and Flavor Chemicals, 1969, Montclair, N.J., USA, the contents of which are hereby incorporated by reference in its entirety, or its more recent versions, or in other works of similar nature.

The proportions in which the compounds of the invention can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the compounds of the invention are typically present at concentrations between about 0.1 and about 10%, or even more, by weight of these compounds relative to the weight of the perfuming composition in which they are incorporated. Far lower concentrations than those mentioned above can be used when the compounds are directly applied for perfuming the various consumer products mentioned above.

The compounds may be used in detergents containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These aspects are described in more detail below.

In addition to the compounds of the invention, the compositions herein may include a detersive surfactant and optionally, one or more additional detergent ingredients, including materials for assisting or enhancing cleaning performance, treatment of the substrate to be cleaned, or to modify the aesthetics of the detergent composition (e.g., perfumes, colorants, dyes, etc.). Non-limiting examples of synthetic detersive surfactants useful herein typically at levels from about 0.5% to about 90%, by weight, include the conventional C₁₋₁₈ alkyl benzene sulfonates (“LAS”) and primary, branch-chain and random C₁₀₋₂₀ alkyl sulfates (“AS”), and the like. Preferred compositions incorporating only synthetic detergents have a detergent level of from about 0.5% to 50%. Compositions containing soap preferably comprise from about 10% to about 90% soap.

The compositions described herein can contain other ingredients such as enzymes, bleaches, fabric softening agents, dye transfer inhibitors, suds suppressors, and chelating agents, all well known within the art.

The compounds of the invention can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and/or fruit juice concentrates.

Artificial colorants that may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts that may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

The beverage may, for example, be a carbonated cola beverage. The pH is generally about 2.8 and the following ingredients can be used to make the syrup for these compositions: Flavor Concentrate, including one or more of the compounds of the invention herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water.

Flavored food and pharmaceutical compositions including one or more of the compounds of the invention can also be prepared. The compounds of the invention can be incorporated into conventional foodstuffs using techniques well known to those of skill in the art. Alternatively, the compounds can be incorporated within polymeric particles, which can, in turn, be dispersed within and/or over a surface of an orally-deliverable matrix material, which is usually a solid or semi-solid substrate. When used in chewable compositions, the compounds of the invention can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

Flavored compositions of the invention may include an orally-deliverable matrix material; a plurality of water insoluble polymeric particles dispersed in the orally-deliverable matrix material, where the polymeric particles individually define networks of internal pores and are non-degradable in the digestive tract; and one or more compounds of the invention entrapped within the internal pore networks. The compounds of the invention are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a compound or a mixture of compounds of the invention to a desired flavor concentration. In one method for producing such gum based products a blade mixer is heated to about 110° F., the gum base is preheated so that it is softened, and the gum base is then added to the mixer and allowed to mix for approximately 30 seconds. The compound or compounds of the invention are then added to the mixer and mixed for a suitable amount of time. The gum can be then removed from the mixer and rolled to stick thickness on waxed paper while warm.

The compounds of the invention may be incorporated into a system that can release a fragrance in a controlled manner. These include substrates such as air fresheners, laundry detergents, fabric softeners, deodorants, lotions, and other household items. The fragrances are generally one or more derivatives of essential oils as described herein, each present in different quantities. U.S. Pat. No. 4,587,129, the contents of which are hereby incorporated by reference in their entirety, describes a method for preparing gel articles that contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy)lower alkyl 2-alkeneoate and a polyethylenically unsaturated crosslinking agent. These materials have continuous slow release properties, i.e. they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

The present invention is illustrated by the following non-limiting examples.

### EXAMPLE 1

Equimolar amounts of amine, (267 grams of oleyl amine) and odorant (152 grams of 2-pentyl-2-cyclopenten-1-one) were mixed by stirring thoroughly and warmed to 50° C. while stirring for 3 to 5 hours, at which time the reaction had gone substantially to completion.

The process of the reaction can be monitored by any suitable means know in the art such as NMR or chromatography eg gas chromatography or thin layer chromatography.

### EXAMPLE 2

Stoichiometric amounts of amine (35 g oleyl amine) and to filbertone (10 grams) were mixed together by stirring at about 50° C. for 3 to 5 hours, until the reaction had gone substantially to completion. When the reaction mixture had cooled (30 minutes), the product was ready for separation.

The separated product had a viscosity of from 50 to 100 cP.

### EXAMPLE 3

A mixture of the amine (oleyl amine) and the musk aldehyde having the formula 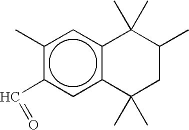
was stirred in a suitable solvent (e.g. ethanol, dipropylene glycol, diisopropyl myristate) until imine formation was substantially complete as judged by thin layer chromatography or NMR. Additives such as acids (e.g., paratoluene sulfonic acid) and dehydrating agents (e.g., molecular sieves/sodium sulfate/magnesium sulfate) may be used to accelerate the reaction. Elevated temperatures can be employed also to improve the condensation. When complete the reaction was worked up in an appropriate manner (e.g., filtering to remove insoluble additives/washing to remove additives) and concentrated to yield the product.

### EXAMPLE 4

The product of Example 2 was dissolved in a fabric conditioner at a concentration at which fragrances are typically found in fabric conditioners. A control fabric conditioner containing filbertone at the same concentration was also produced.

Garments were treated with the two fabric conditioners in a rinse cycle of a domestic washing machine and then dried.

It was found that garments treated with the fabric conditioner containing the product of Example 2 exhibited an odor attributable to filbertone for significantly longer than garments treated with a fabric conditioner containing filbertone itself at the same concentration. Garments treated with the filbertone containing fabric conditioner exhibited an odor attributable to filbertone for up to about one hour while garments treated with a fabric conditioner containing the product of Example 2 exhibited an odor attributable to filbertone for up to about two weeks.

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A pro-odorant or pro-flavorant that is liquid at room temperature and has a relatively low viscosity and has the formula (I): 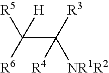
wherein R¹ is H, an aliphatic group or an aromatic group, R² is an aliphatic group or an aromatic group, provided that the total number of carbon atoms in the groups R¹ and R² is 10 or more;
R⁵ is CN, COOH, COOR⁷, CHO or C(O)R⁸
R³, R⁴ and R⁶ are each independently hydrogen or organic moieties which together with R⁵ render a compound of formula R⁵R⁶C═CR³R⁴ a material having odorant or flavourant characteristics and R⁷ and R⁸ are each independently an organic moiety; or
the formula (II)
R⁹R¹⁰C═NR¹¹
wherein R¹¹ has at least 10 carbon atoms and is an aliphatic group or an aromatic group; R⁹ and R¹⁰ are each independently H or organic moieties which together with C═O render a compound of formula R⁹R¹⁰C═O a material having odorant or flavourant characteristics, provided that only one of R⁹ and R¹⁰ is hydrogen;
or the formula (III) 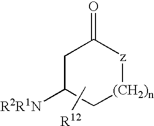
wherein R¹ is H, an aliphatic group or an aromatic group, R² is an aliphatic group or an aromatic group, provided that the total number of carbon atoms in the groups R¹ and R² is 10 or more; Z is CH₂ or O, n is 0 or 1, such that the ring is a 5 or 6 membered ring, R¹² is H, alkyl or alkenyl or alkoxy having up to 10 carbon atoms so as to render a compound of formula 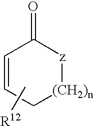
a material having odorant or flavorant characteristics; or
formula (IV): 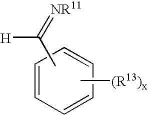
wherein R¹¹ has at least 10 carbon atoms and is an aliphatic group or an aromatic group; the or each R¹³ is independently a straight or branched chain, saturated or unsaturated hydrocarbyl group or alkoxy group having from 1 to 8 carbon atoms or two groups R¹³ together with the carbon atoms to which they are attached form a five or six membered ring which may be saturated or unsaturated (including aromatic) and which may be optionally substituted with from 1 to 3 alkyl groups having from 1 to 6 carbon atoms; and x is from 1 to 5 so as to render a compound of formula 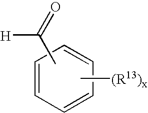
a material having odorant or flavorant characteristics.

2. A compound of formula (I) according to claim 1 having the formula:

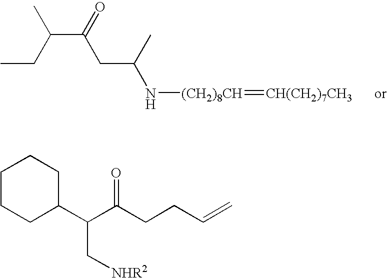

3. A compound of formula (IV) according to claim 1 having the formula:

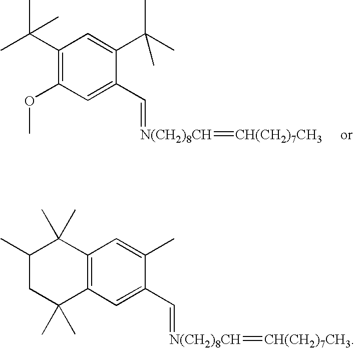

4. A compound according to any one of the preceding claims having a viscosity below about 250 cP, when measured at about 20° C.

5. A compound according to claim 4 having a viscosity below about 100 cP, when measured at about 20° C.

6. A process for producing a compound of formula (I) as defined in claim 1, which comprised reacting a compound of formula (V): 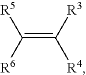
wherein R³, R⁴, R⁵ and R⁶ are each as defined in claim 1 with an amine of formula NHR¹R² wherein R¹ and R² are as defined in claim 1; or
a process for producing a compound of formula (II) as defined in claim 1, which comprised reacting a compound of formula (VI):
R⁹R¹⁰C═O
wherein R⁹ and R¹⁰ are as defined in claim 1 with an amine of formula NH₂R¹¹, wherein R¹¹ is as defined in claim 1; or
a process for producing a compound of formula (III) as defined in claim 1, which comprised reacting a compound of formula (VII): 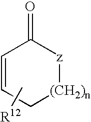
wherein Z is CH₂ or 0, n is 0 or 1, such that the ring is 5- or 6-membered, respectively, and R¹² is as defined in claim 1 with an amine of formula NHR¹R², wherein R¹ and R² are as defined in claim 1; or
a process for producing a compound of formula (IV) as defined in claim 1, which comprised reacting a compound of formula (VIII): 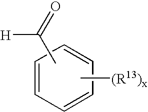
wherein R¹³ and x are as defined in claim 1 with an amine of formula NH₂R¹¹, wherein R¹¹ is as defined in claim 1.

7. A process according to claim 6, wherein the amine is selected from n-dodecylamine, n-tetradecylamine, n-hexadecylamine, n-octadecylamine, oleylamine, cocoalkylamines, soyaalkylamines, tallowalkylamines, hydrogenated tallowalkylamines, di-n-hexylamine, di-n-octylamine, di-n-decylamine, di-n-docecylamine, di-n-tetradecylamine, di-n-hexadecylamine, di-n-octadecylamine, dioleylamine, dicocoalkylamines, disoyaalkylamines, ditallowalkylamines, di-(hydrogenated tallowalkyl)amines, mixed secondary amines such as n-dodecylmethylamine, n-tetradecylmethylamine, n-hexadecylmethylamine, n-octadecylmethylamine, oleylmethylamine, cocoalkylmethylamines, soyaalkylmethylamines, tallowalkylmethylamines, hydrogenated tallowalkylmethylamines, n-decylethylamine, n-dodecylethylamine, n-tetradecylethylamine, n-hexadecylethylamine, n-octadecylethylamine, oleylethylamine, cocoalkylethylamines, soyaalkylethylamines, tallowalkylethylamines, hydrogenated tallowalkylethylamines, branched isomers and derivatives thereof and mixtures thereof.

8. A process according to claim 6, wherein the amine of formula NHR¹R² or NH₂R¹¹ comprises at least one branched or straight chain alkyl or alkenyl group having at least about 10 carbon atoms and is relatively odorless.

9. A process according to claim 7, wherein the amine is oleyl amine.

10. A process according to any one of claims 5 to 8, wherein the compound of formula (V), (VI), (VII) or (VIII) is selected from buccoxime; iso jasmone; methyl beta naphthyl ketone; musk indanone; tonalid/musk plus; alpha-damascone, beta-damascone, delta-damascone, iso-damascone, damascenone, damarose, methyl-dihydrojasmonate, menthone, carvone, camphor, fenchone, alpha-ionone, beta-ionone, gamma-methyl so-called lonone, fleuramone, dihydrojasmone, cis-jasmone, iso-E-Super, methyl-cedrenyl-ketone or methyl-cedrylone, acetophenone, methyl-acetophenone, para-methoxy-acetophenone, methyl-beta-naphtyl-ketone, benzyl-acetone, benzophenone, para-hydroxy-phenyl-butanone, celery ketone or livescone, 6-isopropyldecahydro-2-naphtone, dimethyl-octenone, freskomenthe, 4-(1-ethoxyvinyl)-3,3,5,5,-tetramethyl-cyclohexanone, methyl-heptenone, 2-(2-(4-methyl-3-cyclohexen-1-yl)propyl)cyclopentanone, 1-(p-Menthen-6(2)-yl)-1-propanone, 4-(4-hydroxy-3-methoxyphenyl)-2-butanone, 2-acetyl-3,3-dimethyl-norbornane, 6,7-dihydro-1,1,2,3,3-pentamethyl-4(5H)-indanone, 4-damascol, dulcinyl or cassione, gelsone, hexalon, isocyclemone E, methyl cyclocitrone, methyl-lavender-ketone, orivon, para-tertiary-butyl-cyclohexanone, verdone, delphone, muscone, neobutenone, plicatone, veloutone, 2,4,4,7-tetramethyl-oct-6-en-3-one, tetrameran, adoxal, anisic aldehyde, cymal, ethyl vanillin, florhydral, helional, heliotropin, hydroxycitronellal, koavone, lauric aldehyde, lyral, methyl nonyl acetaldehyde, P. T. bucinal, phenyl acetaldehyde, undecylenic aldehyde, vanillin, 2,6,10-trimethyl-9-undecenal, 3-dodecen-1-al, alpha-n-amyl cinnamic aldehyde, 4-methoxybenzaldehyde, benzaldehyde, 3-(4-tert butylphenyl)-propanal, 2-methyl-3-(para-methoxyphenyl)propanal, 2-methyl-4-(2,6,6-trimethyl-2(1)-cyclohexen-1-yl)butanal, 3-phenyl-2-propenal, cis/trans-3,7-dimethyl-2,6-octadien-1-al, 3,7-dimethyl-6-octen-1-al, [(3,7-dimethyl-6-octenyl)oxy]acetaldehyde, 4-isopropylbenzyaldehyde, 1,2,3,4,5,6,7,8-octahydro-8,8-dimethyl-2-naphthaldehyde, 2,4-dimethyl-3-cyclohexen-1-carboxaldehyde, 2-methyl-3-(isopropylphenyl)propanal, 1-decanal; decyl aldehyde, 2,6-dimethyl-5-heptenal, 4-(tricyclo[5.2.1.0(2,6)]-decylidene-8)-butanal, octahydro-4,7-methano-1H-indenecarboxaldehyde, 3-ethoxy-4-hydroxy benzaldehyde, para-ethyl-alpha, alpha-dimethyl hydrocinnamaldehyde, alpha-methyl-3,4-(methylenedioxy)-hydrocinnamaldehyde, 3,4-methylenedioxybenzaldehyde, alpha-n-hexyl cinnamic aldehyde, m-cymene-7-carboxaldehyde, alpha-methyl phenyl acetaldehyde, 7-hydroxy-3,7-dimethyl octanal, undecenal, 2,4,6-trimethyl-3-cyclohexene-1-carboxaldehyde, 4-(3)(4-methyl-3-pentenyl)-3-cyclohexen-carboxaldehyde, 1-dodecanal, 2,4-dimethyl cyclohexene-3-carboxaldehyde, 4-(4-hydroxy-4-methyl pentyl)-3-cylohexene-1-carboxaldehyde, 7-methoxy-3,7-dimethyloctan-1-al, 2-methyl undecanal, 2-methyl decanal, 1-nonanal, 1-octanal, 2,6,10-trimethyl-5,9-undecadienal, 2-methyl-3-(4-tertbutyl)propanal, dihydrocinnamic aldehyde, 1-methyl-4-(4-methyl-3-pentenyl)-3-cyclo-hexene-1-carboxaldehyde, 5 or 6 methoxyohexahydro-4,7-methanoindan-1 or 2-carboxaldehyde, 3,7-dimethyloctan-1-al, 1-undecanal, 10-undecen-1-al, 4-hydroxy-3-methoxy benzaldehyde, 1-methyl-3-(4-methylpentyl)-3-cyclohexene-carboxaldehyde, 7-hydroxy-3,7-dimethyl-octanal, trans-4-decenal, 2,6-nonadienal, para-tolylacetaldehyde; 4-methylphenylacetaldehyde, 2-methyl-4-(2,6,6-trimethyl-1-cyclohexen-1-yl)-2-butenal, ortho-methoxycinnamic aldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindan-1-carboxaldehyde, 2-methyl octanal, alpha-methyl-4-(1-methyl ethyl)-benzene acetaldehyde, 6,6-dimethyl-2-norpinene-2-propionaldehyde, para methyl phenoxy acetaldehyde, 2-methyl-3-phenyl-2-propen-1-al, 3,5,5-trimethyl hexanal, hexahydro-8,8-dimethyl-2-naphthaldehyde, 3-propyl-bicyclo[2.2.1]hept-5-ene-2-carbaldehyde, 9-decenal, 3-methyl-5-phenyl-1-pentanal, methylnonyl acetaldehyde, hexanal, trans-2-hexenal, 1-para-menthene-q-carboxaldehyde, 2-methyl-2-(para-isopropylphenyl)-propionaldehyde, 1-(2,6,6-trimethyl-2-cyclohexan-1-yl)-2-buten-1-one and/or para-methoxy-acetophenone, undecylenic aldehyde, undecalactone gamma, heliotropin, dodecalactone gamma, para-anisic aldehyde, para-hydroxy-phenyl-butanone, cymal, ionone alpha, damascenone, ionone beta, methyl-nonyl ketone, compounds having the formula: 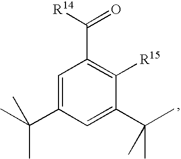
wherein R¹⁴ and R¹⁵ are, independently, H or straight or branched chain alkyl or alkenyl having 1 to 6 carbon atoms; 2,4-di-tertiarybutyl-5-methoxybenzaldehyde; polycyclic musks having the structures: 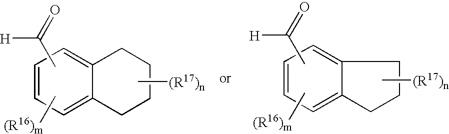
wherein R¹⁶ and R¹⁷ may be the same or different and are a straight or branched chain, saturated or unsaturated hydrocarbyl group; preferably, alkyl or alkenyl, having 1 to 8 carbon atoms, and m is 1 to 4 and n is 1 to 6; 4-methyl-pentan-2-ol-crotonate, 1-cyclohexyl-ethyl-crotonate (Datilat) and hexylcrotonate; butyl pentenoate; ethyl pentenoate; hexyl angelate; hexyl pentenoate; iso-amyl angelate; iso-butyl angelate; iso-amyl pentenoate; iso-byutul pentenoate; methyl allyl pentenoate; methylgeranate; cis-3-hexenylsalicylate; methyl-2-nonenoate; 3,7-dimethyl-6-octenyl-2-methylcrotonate; phenylethyl cinnamate; 3,7-dimethyl-2,6-octadienyl-2-methylcrotonate; methyl-2-nonenoate; 4-methyl-pentan-2-ol-crotonate (Frutinat); 2-cyclopentyl-cyclopentylcrotonate (Pyproprunat); 3,7-dimethyl-2(3), 6-nonadienenitrile (lemonile), tridecene-2-nitrile, 3,12-tridecadienenitrile, 3-methyl-5-phenyl-2-pentenenitrile, 3,7-dimethyl-2,6-octadienenitrile and cinnamylnitrile, filbertone, 2-pentyl-2-cyclopenten-1-one, 2-cyclohexyl-1,6-heptadien-3-one, delta-2-decenolactone, 2-pentyl-2-cyclopenten-1-one, 2-hexyl-2-cyclopenten-1-one, 2-hexen-1,4-lactone, but-2-en-1,4-lactone, 2-decen-1,4-lactone, 2-me-2-pentenoic acid and mixtures thereof.

11. A process according to claim 6 for producing a compound of claim 3, which comprises reacting a compound of formula: 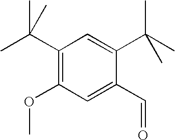
with H₂N(CH₂)₈CH═CH(CH₂)₇CH₃ or reacting a compound of formula 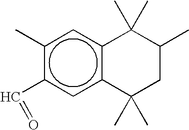
with H₂N(CH₂)₈CH═CH(CH₂)₇CH₃.

12. The use of a compound as defined in any one of claims 1 to 5 as a flavour or fragrance.

13. A substrate treated with a compound as defined in any one of claims 1 to 5.

14. A method for treating a substrate to impart flavorant/fragrance releasing characteristics thereto comprising treating the substrate with a compound as defined in any one of claims 1 to 5.

15. A composition, product, preparation or article having aroma, fragrance or odor releasing characteristics containing a compound or mixture of compounds as defined in any one of claims 1 to 5 optionally in admixture with other perfuming ingredients, solvents, or adjuvants of current use in the art.

16. A composition, product, preparation or article according to claim 15, wherein the compound or mixture of compounds as defined in any one of claims 1 to 5 is present in an amount of at least 30 percent by weight.

17. A composition, product, preparation or article according to claim 16, wherein the compound or mixture of compounds as defined in any one of claims 1 to 4 is present in an amount of at least 60 percent by weight.

18. A composition, product, preparation or article according to any one of claims 15 to 17 in the form of a perfume, fragrance or cologne, a soap, a bath or shower gel, a shampoo or other hair care product, a cosmetic preparation, a body odorant, deodorant or antiperspirant, an air freshener, a liquid or solid fabric detergent or softener, bleach product, disinfectant or an all-purpose household or industrial cleaner.

19. A detergent composition, product, preparation or article according to claim 18, wherein the compound or mixture of compounds as defined in any one of claims 1 to 5 is in admixture with other detergent ingredients, solvents or adjuvants.

20. A bleach composition, product, preparation or article according to claim 18, wherein the compound or mixture of compounds as defined in any one of claims 1 to 5 is in admixture with other bleach ingredients, solvents or adjuvants.

21. A disinfectant composition, product, preparation or article according claim 18, wherein the compound or mixture of compounds as defined in any one of claims 1 to 5 is in admixture with other disinfectant ingredients, solvents or adjuvants.

22. A composition, product, preparation or article according to claim 18 in the form of a body odorant, deodorant or antiperspirant wherein the compound or mixture of compounds is in admixture with other body odorant, deodorant or antiperspirant ingredients, solvents or adjuvants.

23. A composition, product, preparation or article having improved flavor or taste characteristics containing a compound or mixture of compounds as defined in any one of claims 1 to 5.

24. A composition, product, preparation or article according to claim 23 in the form of a beverage, which optionally contains other beverage ingredients, solvents or adjuvants.

25. A composition, product, preparation or article according to claim 23 in the form of a flavoring, which optionally contains other flavoring ingredients, solvents or adjuvants.

26. A composition, product, preparation or article according to claim 23 in the form of a food, which optionally contains other food ingredients, solvents or adjuvants.

27. A composition, product, preparation or article according to claim 23 in the form of a chewing gum, which optionally contains other chewing gum ingredients, solvents or adjuvants.

28. A composition, product, preparation or article according to claim 23 in the form of a pharmaceutical, which optionally contains other pharmaceutical ingredients, solvents or adjuvants.

29. A composition, product, preparation or article according to claim 23 in the form of an orally-deliverable matrix material which may optionally contain other matrix material ingredients, solvents or adjuvants.

30. A method to confer, improve, enhance or modify a taste or flavor property of a composition, product, preparation or article which comprises adding thereto a flavor effective amount of a compound or mixture of compounds as defined in any one of claims 1 to 5.

31. A method according to claim 30, wherein said composition, product, preparation or article is in the form of a beverage, a flavoring, a food, a chewing gum, a pharmaceutical or an orally deliverable matrix.

32. A method to confer, improve, enhance or modify an aroma, fragrance or odor characteristics of a composition, product, preparation or article which comprises adding thereto an aroma, fragrance or odor effective amount of a compound or mixture of compounds as defined in any one of claims 1 to 5.

33. A method according to claim 32 wherein said composition, product, preparation or article is in the form of a perfume, a body odorant, deodorant or antiperspirant, a detergent, a bleach product or a disinfectant.

34. An article of manufacture comprising packaging material and an aroma, odor, fragrance, taste or flavor enhancing agent contained within the packaging material, wherein the agent is effective for the enhancement of the aroma, odor, fragrance, taste or flavor of a composition, preparation, product or article to which it is added, and wherein the packaging material comprises a label which indicates that the agent can be used for enhancing aroma, odor, fragrance, taste or flavor, and wherein the agent is a compound or mixture of compounds as defined in any one of claims 1 to 5.

35. A pro-odorant or pro-flavorant as hereinbefore described.

36. A process or method as hereinbefore described.

37. A composition or article of manufacture as hereinbefore described.
